(19) United States
(12) Patent Application Publication
(10) Pub. No.: US 2018/0220928 A1
(43) Pub. Date: Aug. 9, 2018

(54) SENSOR ASSEMBLIES FOR ELECTROMAGNETIC NAVIGATION SYSTEMS

(71) Applicant: Boston Scientific Scimed Inc., Maple Grove, MN (US)

(72) Inventors: James E. Blood, Shoreview, MN (US); Daniel J. Foster, Lino Lakes, MN (US); Steven J. Meyer, Lake Elmo, MN (US)

(21) Appl. No.: 15/888,697

(22) Filed: Feb. 5, 2018

Related U.S. Application Data
(60) Provisional application No. 62/455,339, filed on Feb. 6, 2017.

(51) Int. Cl. A61B 5/06; G01D 5/14; G01D 5/20; G01D 5/16

(52) U.S. Cl. CPC A61B 5/062; G01D 5/145; A61B 2562/0223; G01D 5/16; G01D 5/2006

(57) ABSTRACT

A sensor assembly comprising a base member extending along a longitudinal axis and including a first portion, a second portion, and a twist section positioned between the first portion and the second portion. The sensor assembly further includes a first magnetic field sensor coupled to the first portion, wherein the first magnetic field sensor has a primary sensing direction aligned with the longitudinal axis, and a second magnetic field sensor coupled to the second portion, wherein the second magnetic field sensor is oriented with respect to the first magnetic field sensor such that the second magnetic field sensor has a primary sensing direction aligned with an axis orthogonal to the longitudinal axis.

### CROSS-REFERENCE TO RELATED APPLICATION

This application claims priority to Provisional Application No. 62/455,339, filed Feb. 6, 2017, which is herein incorporated by reference in its entirety.

### TECHNICAL FIELD

The present disclosure relates to systems, methods, and devices for tracking items. More specifically, the disclosure relates to systems, methods, and devices for electro-magnetically tracking medical devices used in medical procedures.

### BACKGROUND

A variety of systems, methods, and devices can be used to track medical devices. Tracking systems can use externally generated magnetic fields that are sensed by at least one tracking sensor in the tracked medical device. The externally generated magnetic fields provide a fixed frame of reference, and the tracking sensor senses the magnetic fields to determine the location and orientation of the sensor in relation to the fixed frame of reference.

### SUMMARY

In Example 1, a sensor assembly comprising a base member extending along a longitudinal axis and including a first portion, a second portion, and a twist section positioned between the first portion and the second portion, wherein the twist section includes a serpentine shape; a first magnetic field sensor coupled to the first portion, wherein the first magnetic field sensor has a primary sensing direction aligned with the longitudinal axis; and a second magnetic field sensor coupled to the second portion, wherein the second magnetic field sensor is oriented with respect to the first magnetic field sensor such that the second magnetic field sensor has a primary sensing direction aligned with an axis orthogonal to the longitudinal axis.

In Example 2, the sensor assembly of Example 1, further comprising a third magnetic field sensor coupled to the first portion and oriented with respect to the first magnetic field sensor such that the third magnetic field sensor has a primary sensing direction aligned with an axis orthogonal to the longitudinal axis.

In Example 3, a sensor assembly comprising a first base member coupled to a second base member, wherein the first base member extends along a longitudinal axis and includes a first portion, a second portion, and a twist section positioned between the first portion and the second portion; a first magnetic field sensor coupled to the second base member, wherein the first magnetic field sensor has a primary sensing direction aligned with the longitudinal axis; a second magnetic field sensor coupled to the second portion, wherein the second magnetic field sensor is oriented with respect to the first magnetic field sensor such that the second magnetic field sensor has a primary sensing direction aligned with an axis orthogonal to the longitudinal axis.

In Example 4, the sensor assembly of Example 3, further comprising a third magnetic field sensor coupled to the second base member and oriented with respect to the first magnetic field sensor such that the third magnetic field sensor has a primary sensing direction aligned with an axis orthogonal to the longitudinal axis.

In Example 5, the sensor assemblies of any of Examples 1-4, wherein the twist section includes a slit.

In Example 6, the sensor assemblies of any of Examples 1-5, wherein the twist section includes two slits.

In Example 7, the sensor assemblies of any of Examples 1-6, wherein the twist section includes three slits.

In Example 8, the sensor assemblies of any of Examples 1-7, wherein the first, second, and third magnetic field sensors include one of inductive sensing coils, magneto-resistive sensing elements, giant magneto-impedance sensing elements, and flux-gate sensing elements.

In Example 9, the sensor assemblies of Example 8, wherein the magneto-resistive sensing elements include one of anisotropic magneto-resistive sensing elements, giant magneto-resistive sensing elements, tunneling magneto-resistive sensing elements, Hall effect sensing elements, colossal magneto-resistive sensing elements, extraordinary magneto-resistive sensing elements, and spin Hall sensing elements.

In Example 10, the sensor assemblies of any of Examples 1-9, wherein the base members form part of a flex circuit.

In Example 11, the sensor assemblies of any of Examples 1-10, wherein the magnetic field sensors are electrically coupled to conductors.

In Example 12, the sensor assemblies of any of Examples 1-11, wherein the magnetic field sensors are wire bonded to conductors.

In Example 13, the sensor assemblies of any of Examples 1-12, wherein the magnetic field sensors are coupled to conductors via a flip-chip approach.

In Example 14, a method for making the sensor assemblies of any of Examples 1-13, the method comprising twisting the twist section such that the primary sensing directions of the first and second magnetic field sensors are oriented orthogonal to each other.

In Example 15, the method of Example 14, further comprising after twisting the twist section, cutting the base members to form the sensor assemblies of Examples 1-13.

In Example 16, a medical probe including a distal portion having a sensor assembly, wherein the sensor assembly comprises the sensor assemblies of any of Examples 1-15.

In Example 17, a medical system comprising the medical probe according to Example 16; a magnetic field generator configured to generate a multi-dimensional magnetic field in a volume including the medical probe and a patient; and a processor operable to receive outputs from the sensor assembly to determine a position of the sensor assembly within the volume

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a schematic of a tracking system, in accordance with certain embodiments of the present disclosure.

FIG. 2 shows a schematic of a sensor assembly, in accordance with certain embodiments of the present disclosure.

FIG. 3 shows a schematic of a sensor assembly, in accordance with certain embodiments of the present disclosure.

FIG. 4 shows a schematic of a sensor assembly, in accordance with certain embodiments of the present disclosure.

FIG. 5A shows a schematic of an unassembled sensor assembly, in accordance with certain embodiments of the present disclosure.

FIG. 5B shows a schematic of the sensor assembly of 5A in assembled form.

FIG. 6A shows a schematic of a sensor assembly, in accordance with certain embodiments of the present disclosure.

FIG. 6B shows a schematic of the sensor assembly of FIG. 6A before being manufactured to the shape shown in FIG. 6A.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the invention as defined by the appended claims.

### DETAILED DESCRIPTION

During medical procedures, medical devices such as probes (e.g., catheters) are inserted into a patient through the patient's vascular system and/or a catheter lumen. To track the location and orientation of a probe within the patient, probes can be provisioned with magnetic field sensors.

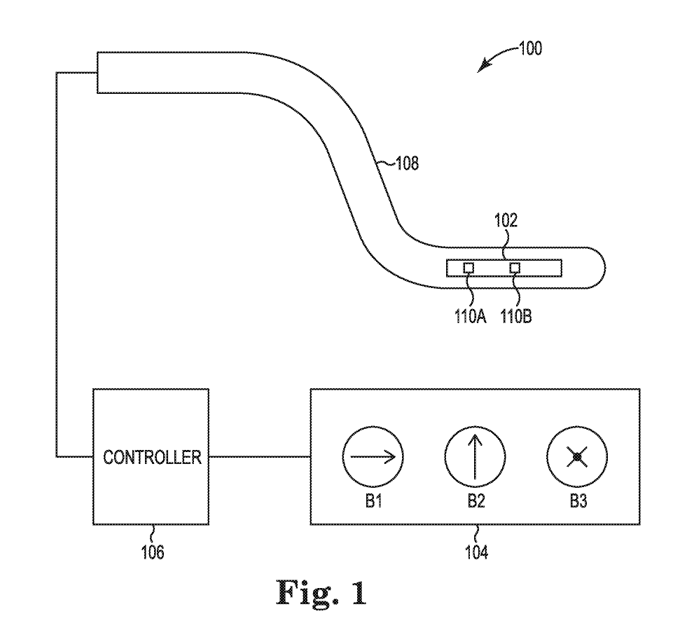

FIG. 1 is a diagram illustrating a tracking system 100 including a sensor assembly 102, magnetic field generator 104, a controller 106, and a probe 108 (e.g., catheter, imaging probe, diagnostic probe). The sensor assembly 102 can be positioned within the probe 108, for example, at a distal end of the probe 108. The tracking system 100 is configured to determine the location and orientation of the sensor assembly 102 and, therefore, the probe 108. Magnetic fields generated by the magnetic field generator 104 provide a frame of reference for the tracking system 100 such that the location and orientation of the sensor assembly 102 is determined relative to the generated magnetic fields. The tracking system 100 can be used in a medical procedure, where the probe 108 is inserted into a patient and the sensor assembly 102 is used to assist with tracking the location of the probe 108 in the patient.

The sensor assembly 102 is communicatively coupled to the controller 106 by a wired or wireless communications path such that the controller 106 sends and receives various signals to and from the sensor assembly 102. The magnetic field generator 104 is configured to generate one or more magnetic fields. For example, the magnetic field generator 104 is configured to generate magnetic fields with components in different directions B1, B2, and B3, as indicated by arrows in FIG. 1. The controller 106 is configured to control the magnetic field generator 104 via a wired or wireless communications path to generate one or more of the magnetic fields to assist with tracking the sensor assembly 102 (and therefore probe 108).

The sensor assembly 102 is configured to sense the generated magnetic fields and provide tracking signals indicating the location and orientation of the sensor assembly 102 in up to six degrees of freedom (i.e., x, y, and z measurements, and pitch, yaw, and roll angles). Generally, the number of degrees of freedom that a tracking system is able to track depends on the number of magnetic field sensors and magnetic field generators. For example, a tracking system with a single magnetic field sensor may not be capable of tracking roll angles and thus are limited to tracking in only five degrees of freedom (i.e., x, y, and z coordinates, and pitch and yaw angles). This is because a magnetic field sensed by a single magnetic field sensor does not change as the single magnetic field sensor is “rolled.” As such, the sensor assembly 102 includes at least two magnetic field sensors, 110A and 110B. The magnetic field sensors can include sensors such as inductive sensing coils and/or various sensing elements such as magneto-resistive (MR) sensing elements (e.g., anisotropic magneto-resistive (AMR) sensing elements, giant magneto-resistive (GMR) sensing elements, tunneling magneto-resistive (TMR) sensing elements, Hall effect sensing elements, colossal magneto-resistive (CMR) sensing elements, extraordinary magneto-resistive (EMR) sensing elements, spin Hall sensing elements, and the like), giant magneto-impedance (GMI) sensing elements, and/or flux-gate sensing elements. In addition, the sensor assembly 102 and/or the probe 108 can feature other types of sensors, such as temperature sensors, ultrasound sensors, etc.

The sensor assembly 102 is configured to sense each of the magnetic fields and provide signals to the controller 106 that correspond to each of the sensed magnetic fields. The controller 106 receives the signals from the sensor assembly 102 via the communications path and determines the position and location of the sensor assembly 102 and probe 108 in relation to the generated magnetic fields.

The magnetic field sensors, 110A and 110B, can be powered by voltages or currents to drive or excite elements of the magnetic field sensors. The magnetic field sensor elements receive the voltage or current and, in response to one or more of the generated magnetic fields, the magnetic field sensor elements generate sensing signals, which are transmitted to the controller 106. The controller 106 is configured to control the amount of voltage or current to the magnetic field sensors and to control the magnetic field generators 104 to generate one or more of the magnetic fields. The controller 106 is further configured to receive the sensing signals from the magnetic field sensors and to determine the location and orientation of the sensor assembly 102 (and therefore probe 108) in relation to the magnetic fields. The controller 106 can be implemented using firmware, integrated circuits, and/or software modules that interact with each other or are combined together. For example, the controller 106 may include computer-readable instructions/code for execution by a processor. Such instructions may be stored on a non-transitory computer-readable medium and transferred to the processor for execution. In general, the controller 106 can be implemented in any form of circuitry suitable for controlling and processing magnetic tracking signals and information.

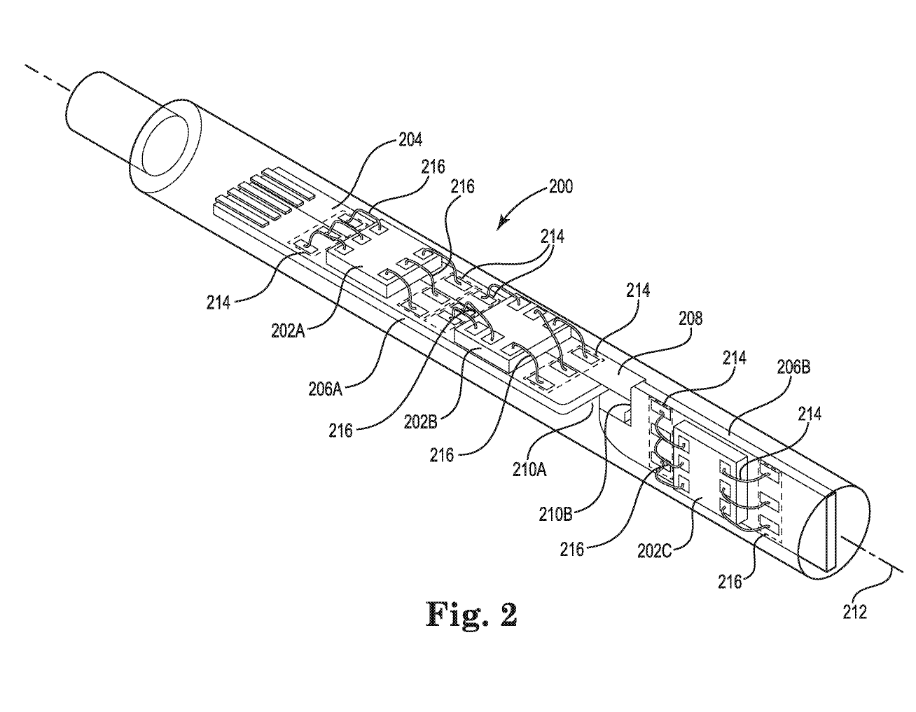

FIG. 2 shows a sensor assembly 200 that can be used in a probe (like the probe 108 in FIG. 1). The sensor assembly 200 includes first, second, and third magnetic field sensors, 202A, 202B, and 202C. The magnetic field sensors 202A, 202B, and 202C can include sensors such as inductive sensing coils and/or various sensing elements such as MR sensing elements (e.g., AMR sensing elements, GMR sensing elements, TMR sensing elements, Hall effect sensing elements, CMR sensing elements, EMR sensing elements, spin Hall sensing elements, and the like), GMI sensing elements, and/or flux-gate sensing elements. The MR sensing elements are configured to sense magnetic fields, like those generated by the magnetic field generator 104 of FIG. 1, and generate a responsive sensing signal. In addition, the sensor assembly 200 can feature other types of sensors, such as temperature sensors, ultrasound sensors, etc. Sensing signals generated by the magnetic field sensors 202A-C can be transmitted from the sensing assembly 200 to a controller, such as the controller 106 of FIG. 1, wirelessly or via one or more conductors.

The first magnetic field sensor 202A, the second magnetic field sensor 202B, and the third magnetic field sensor 202C are shown being positioned on a common sensor assembly substrate 204, which can form part of a flex circuit. In some embodiments, the flex circuit is a single- or double-sided printed circuit board. In some embodiments, the flex circuit is a multi-layer flex circuit. The flex circuit can comprise a flexible substrate (e.g., polyimide, polyester/PET, PEEK, parylene, LCP, PEN, PEI, FEP, and the like), copper clad and/or thin film metallization, and/or laminate materials or liquid dielectrics.

The substrate 204 includes a first portion 206A on which the first and second magnetic field sensors 202A and 202B are positioned and a second portion 206B on which the third magnetic field sensor 202C is positioned. The substrate 204 includes a serpentine flex section 208, which features a first slit 210A and a second slit 210B. The first and second slits 210A and 210B provide increased flexibility such that the flex circuit can be twisted or bent so that the third magnetic field sensor 202C is oriented orthogonal to the first and second magnetic field sensors 202A and 202B. In certain embodiments, the first magnetic field sensor 202A, the second magnetic field sensor 202B, and the third magnetic field sensor 202C are positioned on different sides of the sensor assembly substrate 204. For example, the first magnetic field sensor 202A and the third magnetic field sensor 202C can be positioned on a first side of the sensor assembly substrate 204, and the second magnetic field sensor 202B can be positioned on a second side of the sensor assembly substrate 204 opposite the first side.

The first magnetic field sensor 202A is oriented such that its primary sensing direction is aligned along a longitudinal axis 212 (e.g., X-axis) of the sensor assembly 200. The second magnetic field sensor 202B is oriented such that its primary sensing direction is aligned along an axis (e.g., Y-axis) orthogonal to the longitudinal axis 212. The third magnetic field sensor 202C is oriented such that its primary sensing direction is aligned along an axis (e.g., Z-axis) orthogonal to the X- and Y-axes. The first, second, and third magnetic field sensors, 202A-C, are configured to generate, in response to a magnetic field, responsive sensing signals. The sensing signals are used to determine location and orientation of the sensor assembly 200.

As shown in FIG. 2, the magnetic field sensors, 202A-C, can be electrically coupled to the flex circuit via wire bonding 214 to conductors 216 on the substrate 204. In some embodiments, the magnetic field sensors, 202A-C, are electrically coupled in a flip-chip fashion, solder, fan out, through silicon vias, and the like. The conductors 216 can communicate electrical signals (e.g., sensing signals, power signals) to and from the magnetic field sensors, 202A-C, and the controller 106 of the tracking system 100.

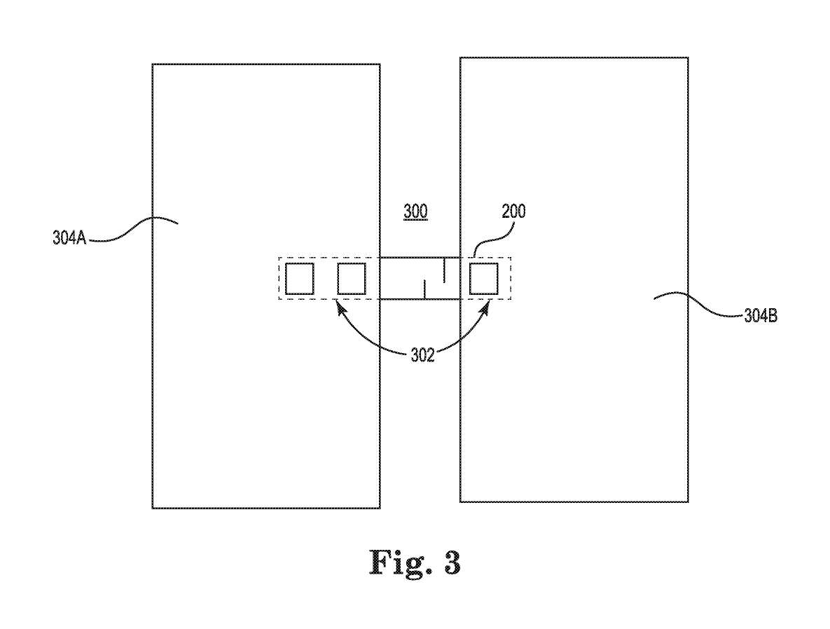

FIG. 3 shows a substrate 300 that is cut to form, for example, the sensor assembly 200. The dotted lines 302 in FIG. 3 represent where the substrate 300 is cut to form the sensor assembly 200. The substrate 300 includes two larger sections (i.e., a first portion 304A and a second portion 304B) that provide increased surface area for a tool to grab so that the sensor assembly 200 can be twisted or bent without over-stressing the sensor assembly 200. Once the sensor assembly 200 is twisted or bent, the two larger sections, 304A and 304B, can be cut along the dotted lines 302 to form the sensor assembly 200.

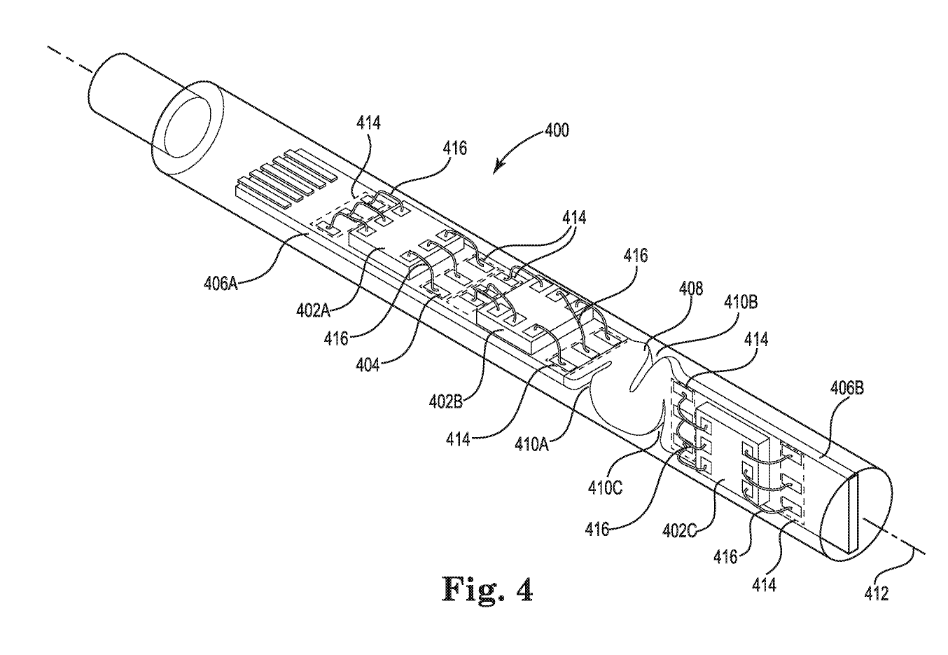

FIG. 4 shows a sensor assembly 400 that can be used in a probe (like the probe 108 in FIG. 1). The sensor assembly 400 includes first, second, and third magnetic field sensors, 402A, 402B, and 402C. The magnetic field sensors 402A, 402B, and 402C can include sensors such as inductive sensing coils and/or various sensing elements such as MR sensing elements (e.g., AMR sensing elements, GMR sensing elements, TMR sensing elements, Hall effect sensing elements, CMR sensing elements, EMR sensing elements, spin Hall sensing elements, and the like), GMI sensing elements, and/or flux-gate sensing elements. The MR sensing elements are configured to sense magnetic fields, like those generated by the magnetic field generator 104 of FIG. 1, and generate a responsive sensing signal. In addition, the sensor assembly 400 can feature other types of sensors, such as temperature sensors, ultrasound sensors, etc. Sensing signals generated by the magnetic field sensors 402A-C can be transmitted from the sensing assembly 400 to a controller, such as the controller 106 of FIG. 1, wirelessly or via one or more conductors.

The first magnetic field sensor 402A, the second magnetic field sensor 402B, and the third magnetic field sensor 402C are shown being positioned on a common sensor assembly substrate 404, which can form part of a flex circuit. In some embodiments, the flex circuit is a single- or double-sided printed circuit board. In some embodiments, the flex circuit is a multi-layer flex circuit. The flex circuit can comprise a flexible substrate (e.g., polyimide, polyester/PET, PEEK, parylene, LCP, PEN, PEI, FEP, and the like), copper clad and/or thin film metallization, and/or laminate materials or liquid dielectrics.

The substrate 404 includes a first portion 406A on which the first and second magnetic field sensors 402A and 402B are positioned and a second portion 406B on which the third magnetic field sensor 402C is positioned. The substrate 404 includes a serpentine flex section 408, which features a first slit 410A, a second slit 410B, and a third slit 410C. The first, second, and third slits 410A-C provide increased flexibility such that the flex circuit can be twisted or bent so that the third magnetic field sensor 402C is oriented orthogonal to the first and second magnetic field sensors 402A and 402B. In some embodiments, the serpentine flex section 408 can include more than three slits.

The first magnetic field sensor 402A is oriented such that its primary sensing direction is aligned along a longitudinal axis 412 (e.g., X-axis) of the sensor assembly 400. The second magnetic field sensor 402B is oriented such that its primary sensing direction is aligned along an axis (e.g., Y-axis) orthogonal to the longitudinal axis 412. The third magnetic field sensor 402C is oriented such that its primary sensing direction is aligned along an axis (e.g., Z-axis) orthogonal to the X- and Y-axes. In certain embodiments, the first magnetic field sensor 402A, the second magnetic field sensor 402B, and the third magnetic field sensor 402C are positioned on different sides of the sensor assembly substrate 404. For example, the first magnetic field sensor 402A and the third magnetic field sensor 402C can be positioned on a first side of the sensor assembly substrate 404, and the second magnetic field sensor 402B can be positioned on a second side of the sensor assembly substrate 404 opposite the first side. The first, second, and third magnetic field sensors, 402A-C, are configured to generate, in response to a magnetic field, responsive sensing signals. The sensing signals are used to determine location and orientation of the sensor assembly 400.

As shown in FIG. 4, the magnetic field sensors, 402A-C, can be electrically coupled to the flex circuit via wire bonding 414 to conductors 416 on the substrate 404. In some embodiments, the magnetic field sensors, 402A-C, are electrically coupled in a flip-chip fashion, solder, fan out, through silicon vias, and the like. The conductors 416 can communicate electrical signals to and from the magnetic field sensors, 402A-C, and the controller 106 of the tracking system 100. The sensor assembly 400 can be formed from a larger substrate such as the substrate shown on FIG. 3 and cut to form the final shape of the sensor assembly 400.

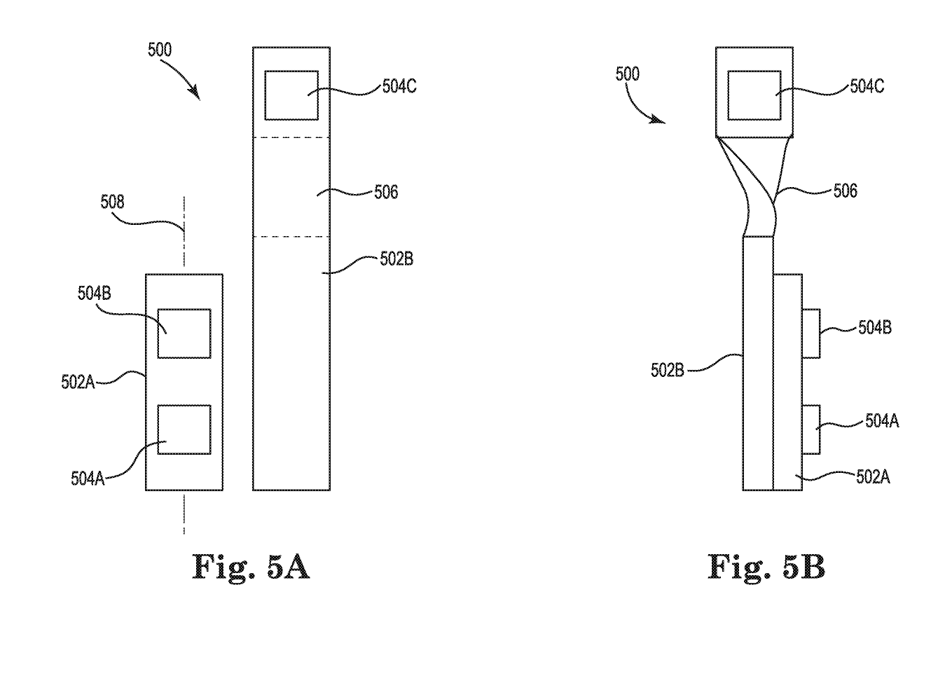

FIGS. 5A and 5B show a sensor assembly 500. FIG. 5A shows an unassembled view of the sensor assembly 500, which includes first substrate 502A and a second substrate 502B of the sensor assembly 500. FIG. 5B shows an assembled view of the sensor assembly 500 where the first and second substrate, 502A and 502B, are mechanically and electrically coupled together. The first substrate 502A includes a first magnetic field sensor 504A and a second magnetic field sensor 504B. The second substrate 502B includes a third magnetic field sensor 504C. The second substrate 502B also includes a flex section 506. The flex section 506 can include slits like the sensor assemblies of FIGS. 2 and 4. In some embodiments, the flex section 506 does not include slits. Before the first and second substrates, 502A-B, are assembled together, the second substrate 502B can be twisted or bent at the flex section 506. In some embodiments, the first and second substrates, 502A-B, for part of one or more flex circuits. In some embodiments, the flex circuits are single- or double-sided printed circuit boards. In some embodiments, the flex circuits are multi-layer flex circuits. The flex circuit can comprise a flexible substrate (e.g., polyimide, polyester/PET, PEEK, parylene, LCP, PEN, PEI, FEP, and the like), copper clad and/or thin film metallization, and/or laminate materials or liquid dielectrics.

When assembled, the first magnetic field sensor 504A is oriented such that its primary sensing direction is aligned along a longitudinal axis 508 (e.g., X-axis) of the sensor assembly 500. The second magnetic field sensor 504B is oriented such that its primary sensing direction is aligned along an axis (e.g., Y-axis) orthogonal to the longitudinal axis 508. The third magnetic field sensor 504C is oriented such that its primary sensing direction is aligned along an axis (e.g., Z-axis) orthogonal to the X- and Y-axes. In certain embodiments, the first magnetic field sensor 502A, the second magnetic field sensor 502B, and the third magnetic field sensor 502C are positioned on different sides of the sensor assembly substrate 504. For example, the first magnetic field sensor 502A and the second magnetic field sensor 502B can be positioned on a first side of the sensor assembly substrate 504, and the third magnetic field sensor 502C can be positioned on a second side of the sensor assembly substrate 504 opposite the first side.

The magnetic field sensors, 504A-C, can be electrically coupled to conductors in a flip-chip fashion, solder, fan out, through silicon vias, and the like. The conductors can communicate electrical signals to and from the magnetic field sensors, 504A-C, and the controller 106 of the tracking system 100. The second substrate 502B of the sensor assembly 500 can be formed from a larger substrate such as the substrate shown on FIG. 3 and cut to form the final shape of the second substrate 5028.

The sensor assembly 500 can be used in a probe (like the probe 108 in FIG. 1). The magnetic field sensors 504A, 504B, and 504C can include sensors such as inductive sensing coils and/or various sensing elements such as MR sensing elements (e.g., AMR sensing elements, GMR sensing elements, TMR sensing elements, Hall effect sensing elements, CMR sensing elements, EMR sensing elements, spin Hall sensing elements, and the like), GMI sensing elements, and/or flux-gate sensing elements. The MR sensing elements are configured to sense magnetic fields, like those generated by the magnetic field generator 104 of FIG. 1, and generate a responsive sensing signal. In addition, the sensor assembly 500 can feature other types of sensors, such as temperature sensors, ultrasound sensors, etc. Sensing signals generated by the magnetic field sensors 504A-C can be transmitted from the sensing assembly 500 to a controller, such as the controller 106 of FIG. 1, wirelessly or via one or more conductors.

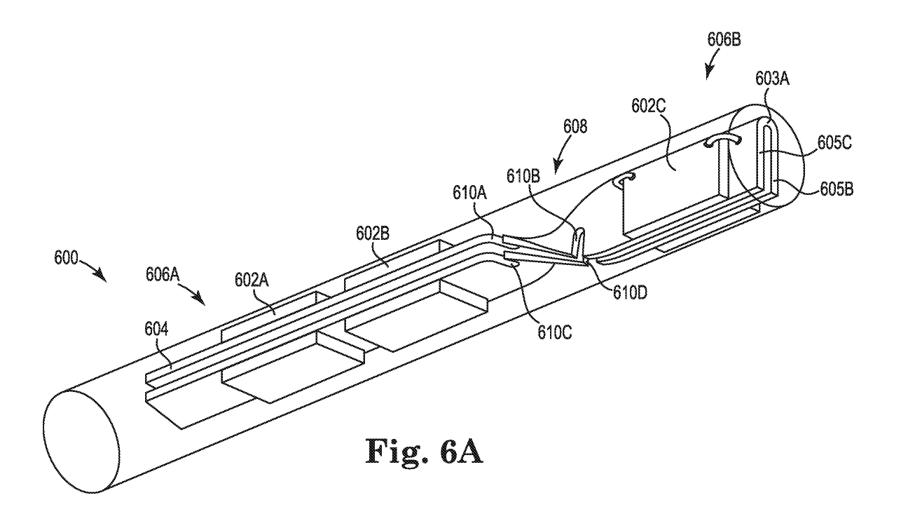

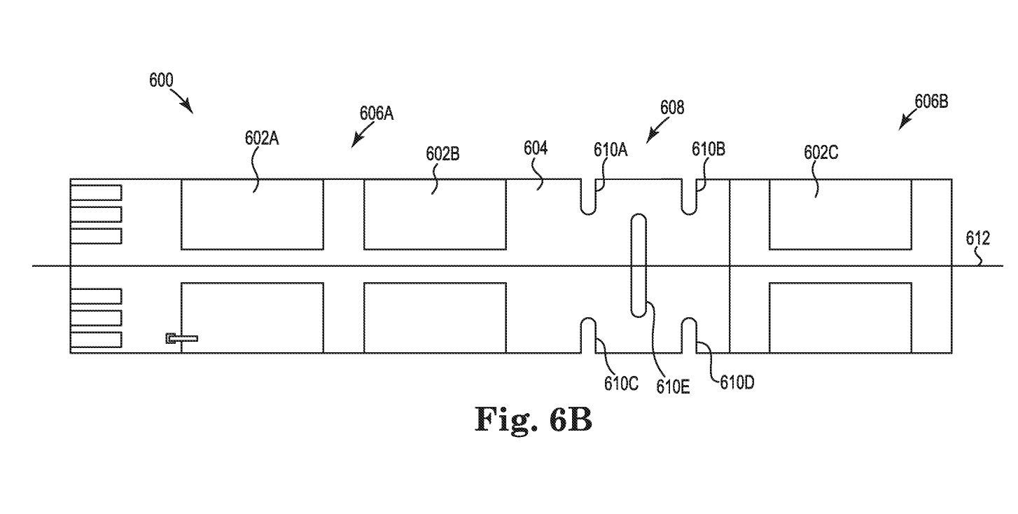

FIG. 6A shows a sensor assembly 600 that can be used in a probe (like the probe 108 in FIG. 1). FIG. 6B shows the sensor assembly 600 before being bent or rolled to the shape shown in FIG. 6A.

The sensor assembly 600 includes first, second, and third magnetic field sensors, 602A, 602B, and 602C. The magnetic field sensors 602A, 602B, and 602C can include sensors such as inductive sensing coils and/or various sensing elements such as MR sensing elements (e.g., AMR sensing elements, GMR sensing elements, TMR sensing elements, Hall effect sensing elements, CMR sensing elements, EMR sensing elements, spin Hall sensing elements, and the like), GMI sensing elements, and/or flux-gate sensing elements. The MR sensing elements are configured to sense magnetic fields, like those generated by the magnetic field generator 104 of FIG. 1, and generate a responsive sensing signal. In addition, the sensor assembly 600 can feature other types of sensors, such as temperature sensors, ultrasound sensors, etc. Sensing signals generated by the magnetic field sensors 602A-C can be transmitted from the sensing assembly 600 to a controller, such as the controller 106 of FIG. 1, wirelessly or via one or more conductors.

The first magnetic field sensor 602A, the second magnetic field sensor 602B, and the third magnetic field sensor 602C are shown being positioned on a common sensor assembly substrate 604, which can form part of a flex circuit. In some embodiments, the flex circuit is a multi-layer flex circuit. The flex circuit can comprise a flexible substrate (e.g., polyimide, polyester/PET, PEEK, parylene, LCP, PEN, PEI, FEP, and the like), copper clad and/or thin film metallization, and/or laminate materials or liquid dielectrics. As shown in FIG. 6B, the substrate 604 can initially be in a planar shape and then bent or rolled into the shape shown in FIG. 6A. In the planar shape, it may be easier to electrically couple the magnetic sensors, etc., to conductors of the sensor assembly 600. In the bent or rolled shape, the substrate 604 has a cross-sectional “C” or “U” shape formed by a bent portion 605A, a first arm portion 605B, and a second arm portion 605C.

The substrate 604 includes a first portion 606A on which the first and second magnetic field sensors 602A and 602B are positioned and a second portion 606B on which the third magnetic field sensor 602C is positioned. The substrate 604 includes a twist section 608, which features a first slit 610A, a second slit 610B, a third slit 610C, a fourth slit 610D, and a fifth slit 610E. The first, second, third, fourth, and fifth slits 610A-E provide increased flexibility such that the flex circuit can be twisted or bent so that the third magnetic field sensor 602C is oriented orthogonal to the first and second magnetic field sensors 602A and 602B. As shown in FIG. 6B, the first through fourth slits, 610A-D, extend inwards from a side of the substrate 604. The fifth slit 610E is shown as an aperture at or near a middle of the substrate 604. In certain embodiments, all slits extend inward from a side of the substrate 604. In certain embodiments, additional slits are positioned as apertures within the substrate 604. In certain embodiments, additional or fewer slits are used to provide increased flexibility such that the flex circuit can be twisted or bent. In certain embodiments, like that shown in FIG. 6A, the twist section 608 is bent or rolled such that the bent portion 605A, the first arm portion 605B, and the second arm portion 605C extend along the substrate 604 including the first portion 606A, the second portion 606B, and the twist section 608. In other embodiments, only the first portion 606A and the second portion 606B are bent or rolled such that the twist section 608 is not bent or rolled.

The first magnetic field sensor 602A is oriented such that its primary sensing direction is aligned along a longitudinal axis 612 (e.g., X-axis) of the sensor assembly 600. The second magnetic field sensor 602B is oriented such that its primary sensing direction is aligned along an axis (e.g., Y-axis) orthogonal to the longitudinal axis 612. The third magnetic field sensor 602C is oriented such that its primary sensing direction is aligned along an axis (e.g., Z-axis) orthogonal to the X- and Y-axes. In certain embodiments, the first magnetic field sensor 602A, the second magnetic field sensor 602B, and the third magnetic field sensor 602C and/or circuitry (e.g., application-specific integrated circuits, diodes, capacitors, and the like) associated with the magnetic field sensors are positioned on different sides (e.g., the first arm portion 605B or the second arm portion 605C) of the sensor assembly substrate 604. For example, as shown in FIG. 6A, the first magnetic field sensor 602A, the second magnetic field sensor 602B, and the third magnetic field sensor 602C are positioned on a first side (e.g., the first arm portion 605B) of the sensor assembly substrate 604 while associated circuitry is positioned on a second side (e.g., the second arm portion 605C) of the sensor assembly substrate 604 opposite the first side. The first, second, and third magnetic field sensors, 602A-C, are configured to generate, in response to a magnetic field, responsive sensing signals. The sensing signals are used to determine location and orientation of the sensor assembly 600.

The magnetic field sensors, 602A-C, can be electrically coupled to the flex circuit via wire bonding to conductors on the substrate 604. In some embodiments, the magnetic field sensors, 602A-C, are electrically coupled in a flip-chip fashion, solder, fan out, through silicon vias, and the like. The conductors can communicate electrical signals to and from the magnetic field sensors, 602A-C, and the controller 106 of the tracking system 100. The sensor assembly 600 can be formed from a larger substrate such as the substrate shown on FIG. 3 and cut to form the final shape of the sensor assembly 600.

It should be noted that, for simplicity and ease of understanding, the elements described above and shown in the figures are not drawn to scale and may omit certain features. As such, the drawings do not necessarily indicate the relative sizes of the elements or the non-existence of other features.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A sensor assembly comprising:
a base member extending along a longitudinal axis and including a first portion, a second portion, and a twist section positioned between the first portion and the second portion, wherein the twist section includes a serpentine shape;
a first magnetic field sensor coupled to the first portion, wherein the first magnetic field sensor has a primary sensing direction aligned with the longitudinal axis; and
a second magnetic field sensor coupled to the second portion, wherein the second magnetic field sensor is oriented with respect to the first magnetic field sensor such that the second magnetic field sensor has a primary sensing direction aligned with an axis orthogonal to the longitudinal axis.

2. The sensor assembly of claim 1, further comprising:
a third magnetic field sensor coupled to the first portion and oriented with respect to the first magnetic field sensor such that the third magnetic field sensor has a primary sensing direction aligned with an axis orthogonal to the longitudinal axis.

3. The sensor assembly of claim 2, wherein the first, second, and third magnetic field sensors include one of inductive sensing coils, magneto-resistive sensing elements, giant magneto-impedance sensing elements, and flux-gate sensing elements.

4. The sensor assembly of claim 3, wherein the magneto-resistive sensing elements include one of anisotropic magneto-resistive sensing elements, giant magneto-resistive sensing elements, tunneling magneto-resistive sensing elements, Hall effect sensing elements, colossal magneto-resistive sensing elements, extraordinary magneto-resistive sensing elements, and spin Hall sensing elements.

5. The sensor assembly of claim 1, wherein the twist section includes a slit.

6. The sensor assembly of claim 1, wherein the twist section includes two slits.

7. The sensor assembly of claim 1, wherein the twist section includes three slits.

8. The sensor assembly of claim 1, wherein the base member includes a bent portion and a first arm portion and a second arm portion extending from the bent portion.

9. The sensor assembly of claim 8, wherein the bent portion, the first arm portion, and the second arm portion form a substantially U- or C-shaped cross section.

10. The sensor assembly of claim 8, wherein twist section includes a plurality of slits extending inwards from a side of the base member, and wherein the twist section further includes an aperture.

11. A sensor assembly comprising:
a first base member coupled to a second base member, wherein the first base member extends along a longitudinal axis and includes a first portion, a second portion, and a twist section positioned between the first portion and the second portion;
a first magnetic field sensor coupled to the second base member, wherein the first magnetic field sensor has a primary sensing direction aligned with the longitudinal axis;
a second magnetic field sensor coupled to the second portion, wherein the second magnetic field sensor is oriented with respect to the first magnetic field sensor such that the second magnetic field sensor has a primary sensing direction aligned with an axis orthogonal to the longitudinal axis.

12. The sensor assembly of claim 10, further comprising:
a third magnetic field sensor coupled to the second base member and oriented with respect to the first magnetic field sensor such that the third magnetic field sensor has a primary sensing direction aligned with an axis orthogonal to the longitudinal axis.

13. The sensor assembly claim 10, wherein the twist section includes a slit.

14. The sensor assembly of claim 10, wherein the twist section includes two slits.

15. The sensor assembly of claim 10, wherein the first, second, and third magnetic field sensors include one of inductive sensing coils, magneto-resistive sensing elements, giant magneto-impedance sensing elements, and flux-gate sensing elements.

16. The sensor assembly of claim 10, wherein the first base member includes a bent portion and a first arm portion and a second arm portion extending from the bent portion.

17. The sensor assembly of claim 16, wherein the bent portion, the first arm portion, and the second arm portion form a substantially U- or C-shaped cross section.

18. A method for making the a sensor assembly having a base member extending along a longitudinal axis and including a first portion, a second portion, and a twist section positioned between the first portion and the second portion, the method comprising:
coupling a first magnetic field sensor to the first portion, the first magnetic field sensor having a primary sensing direction;
coupling a second magnetic field sensor to the second portion, the second magnetic field sensor a primary sensing direction; and
twisting the twist section such that the primary sensing directions of the first and second magnetic field sensors are oriented orthogonal to each other.

19. The method of claim 18, further comprising:
before twisting the twist section, bending the base member such that the base member has a cross-section with a bent portion and a first arm portion and a second arm portion extending from the bent portion.

20. The method of claim 18, further comprising:
after twisting the twist section, cutting the base member to form a sensor assembly.
